(19) United States
(12) Patent Application Publication
(10) Pub. No.: US 2025/0299788 A1
(43) Pub. Date: Sep. 25, 2025

(54) APPARATUS FOR OPTIMIZING A LABORATORY SCHEDULING CONTROL

(71) Applicant: BASF SE, Ludwigshafen am Rhein (DE)

(72) Inventors: Astrid NIEDERLE, Freeport, TX (US); Behrang SHAFEI, Ludwigshafen am Rhein (DE)

(21) Appl. No.: 18/863,248

(22) PCT Filed: May 3, 2023
(86) PCT No.: PCT/EP2023/061614

§ 371 Date: Nov. 5, 2024

(30) Foreign Application Priority Data
May 5, 2022 (EP) 22171750.7

(51) Int. Cl. G16H 10/40

(52) U.S. Cl. CPC G16H 10/40

(57) ABSTRACT

The invention refers to an apparatus 800 for optimizing a laboratory scheduling control. A providing unit 801 provides a probe list including probe data. The probe data comprises probe identifications and tasks. A task is indicative of operations to be performed by a laboratory equipment on the probe. A formulation unit 802 formulates a scheduling problem based on the probe list such that an optimization of the scheduling problem results in an optimization of a scheduling of the tasks. An interface unit 803 interfaces with a quantum computer 810 for utilizing quantum computing for optimizing the scheduling problem and receiving a result of the quantum computation indicative of the optimized scheduling problem. A determination unit 804 determines a schedule for the probe list based on the received result. A control signal determination unit 805 determines a control signal for controlling the laboratory equipment 820 based on the determined schedule.

### FIELD OF INVENTION

The invention relates to an apparatus, a method, and a computer program product for optimizing a laboratory scheduling control. Further, the invention refers to a quantum computer system, a laboratory system, and a scheduling determination system comprising the apparatus.

### BACKGROUND OF THE INVENTION

In a modern laboratory environment, it is often necessary to perform a plurality of tests or analyses steps on a wide variety of different probes in a plurality of predetermined sequences. Moreover, in a modern laboratory environment a high level of automation is preferred in order to decrease the costs of laboratory tests and analyses but also in order to increase the efficiency of performing such tests and analyses. For example, as has been reasonably shown by the COVID pandemic, it can be important to analyse and test as many probes as possible as fast as possible. However, even in an automatized environment it can be difficult to determine a schedule for the respective tests and analyses steps performed on the probes such that the laboratory equipment is optimally utilized and provides the tests and analyses results for each of the probes as fast as possible. In particular, determining such a schedule is a very time consuming and computational resource intensive task that in many cases is still mainly based on the input of an experienced user. Thus, it would be advantages to allow for a less time consuming, less computational resource intensive and more objective scheduling of task in an automatic laboratory environment, in particular, to allow for a more efficient usage of available laboratory equipment and to increase the level of laboratory automation.

### SUMMARY OF THE INVENTION

It is an object of the present invention to provide an apparatus, a method and a computing program product that allow for a less time consuming, less computational resource intensive and more objective optimization of a laboratory scheduling in an automatic laboratory environment. Moreover, the apparatus, method and computing program product allow for a more efficient usage of available laboratory equipment and for an increase in the level of laboratory automation. Further, it is an object of the invention to provide systems utilizing the apparatus, method and/or computer program product for allowing for such an optimization of a laboratory scheduling.

In a first aspect of the invention an apparatus for optimizing a laboratory scheduling control is presented, wherein the apparatus comprises a) a probe list providing unit for providing a probe list including probe data for a plurality of probes, wherein the probe data for a probe comprises a probe identification and a task associated with the probe identification, wherein a task is indicative of one or more operations to be performed with a predetermined timing by a laboratory equipment on the probe, b) a scheduling problem formulation unit for formulating a scheduling problem based on the probe list, wherein the scheduling problem is formulated such that an optimization of the scheduling problem results in an optimization of a scheduling of the tasks associated with the plurality of probes, c) a quantum computer interface unit for interfacing with a quantum computer for utilizing quantum computing for optimizing the scheduling problem and receiving a result of the quantum computation from the quantum computer indicative of the optimized scheduling problem, d) a schedule determination unit for determining a schedule for the probe list based on the received result of the quantum computation, and e) a control signal determination unit for determining a control signal for controlling the laboratory equipment based on the determined schedule.

Since the apparatus comprises a quantum computer interface unit that allows for interfacing with a quantum computer for utilizing quantum computing for optimizing the scheduling problem and receiving a result of the quantum computation from the quantum computer indicative of an optimized scheduling problem and since the scheduling determination unit determines a schedule based on the received result of the quantum computation the advantages of quantum computation in particular in optimization tasks can be exploited to optimize even complex scheduling problems with a high number of tasks and probes to be scheduled. Moreover, utilizing a quantum computer for the calculation of at least parts of the scheduling problem allows to decrease the computational time and necessary computational resources. Furthermore, since even very complex tasks can be solved by utilizing a quantum computer the influence of an experienced user can be decreased or is not even necessary anymore allowing for an objectification of the scheduling. Thus, the apparatus allows for an optimization of a laboratory scheduling in a less time consuming manner, with using less computational resources providing a more objective result. Furthermore, since solutions for more complex problems and a more objective result can be achieved the laboratory equipment can be utilized more efficient and the level of automation increased. Furthermore, downtimes of the laboratory equipment caused by a rescheduling of tasks can be decreased, due to the decreased calculation time for rescheduling tasks.

Generally, the apparatus can be realized in form of software or hardware or a combination thereof, wherein the hardware can refer to any known dedicated or general classical computer hardware. For example, the apparatus can be realized as any known computational device, like a PC. However, the apparatus can also be realized as a cloud environment, computational network, etc., such that at least parts of the apparatus can also be realized as a network solution and thus can be spread over a plurality of computation devices.

The probe list providing unit is adapted to provide a probe list. In particular, the probe list providing unit can refer to a storage unit in which the probe list is already stored. However, the probe list providing unit can also comprise or refer to an input unit which can be utilized, for instance, by a user, to indicate a respective probe list. Moreover, the probe list providing unit can be realized as or communicatively coupled to a scanning device that is adapted to read information provided on the probe itself. For example, the probes can be provided with a barcode on which information referring to the probe is stored and readable for a respective scanning device.

Generally, a scheduling in the context of a laboratory environment refers to a specification indicating which laboratory equipment hast to perform which laboratory operation on which probe in which time slot or at which time. Thus, the laboratory schedule defines the course of actions performed by the laboratory equipment. A controlling performed based on the determined laboratory schedule causes the laboratory equipment thus to perform the predetermined laboratory schedule.

The probe list includes probe data for a plurality of probes. Generally, a probe can refer to any kind of material on which a test procedure or analysis should be performed by the laboratory equipment of a laboratory. For example, the probe can refer to a medical probe like a tissue probe, a blood probe, etc. Moreover, a probe can also refer to any other solid or fluid chemical, mixture or material. For example, the probe can also refer to an environmental probe, like a soil, water or biological material sample. In particular, it is preferred that the probe refers to a sample of a new chemical or material, for example, of a new polymer, medical formulation, or a metal on which respective test procedures, in order to determine a technical applicability, are to be performed. The respective probe can be provided in form of any suitable sample container that allows to hold the sample and is configured to be utilized by the laboratory equipment, for example, to be inserted into a tray or other holding device of the laboratory equipment. Moreover, in an embodiment, a task associated with a probe can also refer to first mixing the probe, for example, from a plurality of other substances in a mixing step. Thus a probe can also refer to a sample that in a first task has to be assembled before further tasks are performed on the probe.

The probe data of a probe comprises a probe identification and further at least one task associated with the probe identification. Generally, the probe identification can refer to any kind of identification that allows to unambiguously identify a specific probe. For example, the probe identification can refer to an identification number of the probe that is also provided, for example, on the probe container, in particular, in form of a barcode, QR-code or any other known computational readable identification marker. However, probe identification can also refer to a specific position of the probe, for example, on a probe tray containing a plurality of probes. In such a case the probe identification can refer, for instance, to specifying the row and column in which the probe can be found in the probe tray. Moreover, the prove identification can also refer to an RFID chip identity of an RFID chip utilized for marking the probe. The at least one task associated with the probe identification and thus with the probe is indicative of one or more operations to be performed with a predetermined timing by a laboratory equipment on the probe. In particular, a task can refer to one or more analysis steps or test procedures to be performed on the probe, wherein the one or more operations are necessary for performing the analysis or test procedure on the probe. The operations can refer, for example, to depleting operations, chemical adding operations, mixing operations, pipetting operations, measuring operations, heating or cooling operations, etc. For example, a task referring to an analysis of a probe can comprise a first operation of depleting the probe, then adding a specific chemical to the probe, then mixing the probe with the chemical, then pipetting and removing parts of the mixed probe and heating the removed part of the mixed probe followed by a predetermined measurement, for example an optical measurement, of the probe. Generally, each of such operations belonging to one task have to be fulfilled with a respective timing. The respective timing can refer to a sequence of the operations and further, if necessary, also to a minimum or a maximum time interval between specific operations. For example, it can be necessary for a task to wait a certain minimum time after one operation before performing the next operation or to perform an operation before a specific maximum time span has expired. Generally, the tasks provided as part of the probe data can be provided in any form that allows to identify the one or more operations that are to be performed as part of the tasks by the laboratory equipment on the probe. For example, for already known tasks the tasks can also be provided in form of an identification, wherein the identification allows to derive the respective operations and the respective predetermined timing between the operations. For example, a plurality of known tasks with respective tasks identifications can be stored together with the respective one or more operations and predetermined timings on a task storage which can be accessed to determine the respective operations of a task. However, the tasks can also be provided in the probe data directly by specifying the one or more operations and the respective timing between the one or more operations. This is in particular advantageous if new tasks, for instance, new analyses or test procedures are introduced. The probe data can generally be provided in any known form that allows an association between the probe identification and thus the probe and the respective task. For example, the probe data can refer to a list of probe identifications and associated tasks or can refer to any other form, for instance, to a table or a matrix.

The scheduling problem formulation unit is configured to formulate a scheduling problem based on the probe list. Generally, the scheduling problem formulation unit can be configured to automatically formulate a scheduling problem, for instance, based on one or more predetermined rules or functions that determine the formulation of the scheduling problem based on the probe list. For example, a general mathematical formulation of a scheduling problem can be utilized by the scheduling problem formulation unit and the information provided by the probe list can be inserted in the general mathematical formulation of the scheduling problem based on predetermined rules. However, the scheduling problem formulation unit can also be configured to allow to formulate the scheduling problem in a user machine interaction process, for example, by providing the user with different possible mathematical formulations of a scheduling problem and allowing the user to select the mathematical formulation of the problem to be utilized to insert the information provided by the probe list. Moreover, an automatically formulate scheduling problem can be provided by the scheduling problem formulation unit to a user for verification or for allowing the user to provide amendments to the formulated scheduling problem.

Generally, the scheduling problem is formulated such that a result of an optimization of the scheduling problem is indicative of an optimization of a scheduling of the tasks, in particular, the one or more operations of the tasks, associated with the plurality of probes. Accordingly, the scheduling problem can also be regarded as referring to an optimization problem. Generally, the scheduling problem can be formulated in any form or representation that allows to determine quantities describing the scheduling problem and the form of interaction between these quantities. Preferably, the scheduling problem is provided in form of a mathematical description of the scheduling problem. However, a scheduling problem can also be described in any other form of notation, for instance, that allows to derive a mathematical formulation of the scheduling problem.

Generally, the optimization problem, i.e. the scheduling problem, refers to an optimization, i.e. minimizing or maximizing, of a functional relation between different quantities. The respective quantities can refer to variables that can be varied during the optimization of the problem. Further, the quantities can refer to fixed or predetermined quantities that are not varied during the optimization of the problem and are set, for example, by constraints. Moreover, the quantities can further refer to one or more quantities which are minimized or maximized during the optimization of the problem referred to as optimization quantities. In particular, the optimization of the optimization problem is performed with respect to one or more variables, i.e. with respect to one or more quantities that can be varied during the optimization in order to minimize or maximize the respectively to be optimized quantity. Such a variable can refer to a scalar variable, i.e. to a quantity that can take on only one value at a time, or can refer to a higher-dimensional variable, in particular, to a set of scalar quantities that can take one value at a time, for instance, can refer to a vector or a matrix. Moreover, the variable can refer also to a part of a higher-dimensional quantity, for instance, can refer to one or more scalar quantities of a matrix or vector. Generally, the optimization problem, i.e. the scheduling problem, can be provided such that the respective variables of the optimization problem can take on any value. However, in a preferred embodiment, the optimization problem is adapted such that the one or more variables are binary variables, i.e. can only take on two values, for example, the values 0 and 1 or 0 and −1 or 1 and −1. This is particular preferred for the parts of the optimization problem calculated on a quantum computer. In a more general case, it is preferred that the optimization problem is adapted such that the variables can take on any integer value. The optimization of the optimization problem, i.e. the scheduling problem, refers to minimizing or maximizing one or more optimization quantities. Preferably, at least one of the optimization quantities refers to a time in which the operations of the tasks associated with the probes of the probe list are performed, wherein this time is minimized during the optimization of the scheduling problem. The variables can refer, for instance, to timeslots at which respective operations are performed on a specific probe. A result of the optimization can thus refer to an indication which operation should be performed on which probe in which timeslot to minimize the time needed to perform all operations indicated by the tasks in the probe list. However, also other or additional optimization quantities can be defined. For example, an optimization quantity can also be the individual time needed for performing operations of the tasks associated with a specific probe, wherein this individual time for each or a selected subset of probes is minimized during the optimization of the scheduling problem. Generally, if more than one optimization quantity is defined that should be optimized during the optimization of the scheduling problem, in most cases it is not possible to optimize one quantity with also influencing the other quantity such that respective optimization results refer to Paretooptimal solutions. Thus, in such a case the scheduling problem can be formulated such that is allows to determined one or more Paretooptimal solutions for the scheduling, wherein based on the determined one or more Paretooptimal solutions a respective scheduling plan can be selected, for instance, by a user, or according to predetermined criteria. The quantum computer interface unit is adapted to interface with a quantum computer for utilizing quantum computing for optimizing the scheduling problem. Generally, the quantum computing can be utilized for optimizing and thus solving the scheduling problem but can also be utilized for calculating only parts of the scheduling problem that can then be utilized for the optimization of the scheduling problem, for example, on a classical computer. Preferably, the quantum computer interface unit is configured for sending at least a part of the scheduling problem to the quantum computer for calculating the at least a part of the scheduling problem utilizing quantum computation.

Quantum computation can be defined as a computation method that is based on a controlled manipulation of quantum elements adhering to the physics of quantum mechanics for performing computational operations. Thus, quantum computation allows to use quantum effects, such as superposition and entanglement. This allows to perform certain computations more efficiently than classical digital computers. In contrast thereto classical computation on classical computing devices can be defined as a computation method that uses processors which are based on transistors that perform computational operations. A quantum computer may be configured for performing a quantum computation. In particular, the quantum computer may comprise one or more quantum elements adhering to the physics of quantum mechanics and means for manipulating the one or more quantum elements for performing computation operations, i.e. for performing a quantum computation. Thus, a quantum computer may be a computer for performing computation operations based on quantum mechanical effects.

The utilized quantum computer can refer to any form of quantum computer. A quantum computer is generally adapted to perform quantum manipulations of respective quantum elements based on control signals for determining a solution of a problem. Quantum manipulations can refer to any manipulations that are performed directly or indirectly on elements of the quantum computer that realize a quantum mechanical description of the to be calculated problem, i.e., that can be described with respect to the quantum mechanical rules instead of the classical physics. In case of a gate-based quantum computer the quantum manipulations are defined as quantum operations that refer to a series of defined direct quantum manipulations of the quantum elements. However, in case of a quantum annealer the quantum manipulations refer to a more indirect manipulation. For example, after the preparation of the initial state of the quantum elements an external field, for example, a magnetic field, is slowly and continuously changed for evolving the states of the quantum elements in the external field into an end state.

Generally, although an element of the quantum computer can be utilized to realize the quantum mechanical description of a problem, i.e. can be described with the quantum mechanical rules, the element itself does not necessarily have to refer to a quantum mechanical system, e.g. an atom or ion. Preferably, the quantum manipulations comprise all operations that directly or indirectly can influence the states of quantum elements, i.e., qubits, of the quantum computer. In particular, the utilized quantum computer can refer to an adiabatic quantum computing system, a quantum annealing system and/or gate-based computing system.

Further, the quantum computer interface unit is adapted to receive a result of the quantum computation from the quantum computer indicative of the optimized scheduling problem. In particular, the result of the quantum computation refers to a result of the calculation of at least a part of the scheduling problem performed on the quantum computer. The respective result is thus directly indicative of the optimized scheduling problem, for instance, directly refers to a solution of the optimized scheduling problem or can be utilized in a calculation of the optimized scheduling problem, for instance, performed on a classical computing device.

The schedule determination unit is then adapted to determine a schedule for the provided probe list based on the received result of the quantum computation. For example, as described above, if the result of the quantum computation refers only to a calculation of a part of the scheduling problem the scheduled determination unit can be adapted to perform or to utilize a calculation of the scheduling problem based on the received result. Moreover, if the received result already refers to the optimized scheduling problem the schedule determination unit can be configured to interpret the optimized scheduling problem to determine the specific schedule of the probe list. For example, a result for the calculation of the optimized scheduling problem can refer to a matrix indicating which operation for which probe is to be performed in which time slot. The scheduling determination unit can then be adapted based on the received matrix to identify, for instance, from the position in the matrix, the respective probe identification the respective operation and the respective time slot on which it should be performed and to generate a list or a table determining for each probe of the probe list in which time slot a respective operation of a respective task are to be performed by which laboratory equipment. Such a list or table can then be regarded as a schedule for the probe list. However, the schedule probe list can also be provided in other digital or analog format that allows to derive the schedule for each probe from the format.

The control signal determination unit is then adapted to determine a control signal for controlling the laboratory equipment based on the determined schedule. Generally, the control signal can be provided in any kind of format that can be interpreted by the laboratory equipment to perform the determined schedule. For example, the control signal can refer to a representation of the schedule that is interpretable by a laboratory management system such that the respective laboratory equipment is controlled in accordance with the schedule. However, the control signal can also directly comprise the signals that allow a controlling of the laboratory equipment, for example, that allow the starting and/or stopping of a mixer, the movement of a robotic system for placing one or more probes at specific locations, the increasing and/or decreasing of the temperature in a heater, the vibration of a vibration plate, etc.

In an embodiment the quantum computer interface unit further comprises a scheduling problem preparation unit for preparing at least a part of the scheduling problem such that the optimization of the scheduling problem is performable utilizing a quantum computation. Generally, the scheduling problem preparation unit can be part of the same hardware and/or software utilized for realizing the apparatus. However, the scheduling problem preparation unit can also be realized in form of a hardware and/or software that is communicatively coupled with the quantum computer interface unit of the apparatus without being itself part of the same hardware and/or software of the quantum computer interface unit. For example, the problem list providing list, the scheduling problem formulation unit, the schedule determination unit and the control signal determination unit can be realized as part of a first web service, wherein the quantum computer interface unit provides an interface to the quantum computer via the scheduling problem preparation unit provided as another web service.

Preferably, preparing at least a part of the scheduling problem such that the optimization of the scheduling problem is performable utilizing a quantum computation comprises determining control operations for controlling the quantum computer to perform the quantum computation of at least a part of the scheduling problem. In particular, the preparation comprises transforming the scheduling problem to quantum operations that can be prepared and performed on a specific quantum computer based on respective control signals causing a manipulation of the quantum elements of the quantum computer in accordance with the determined quantum operations. Moreover, the preparation of the at least a part of the scheduling problem can refer to any kind of preparation and/or transformation of the scheduling problem such that the scheduling problem is performable utilizing a quantum computation. This embodiment is in particular preferred if the scheduling problem formulation unit is not adapted to directly provide the scheduling problem such that it is calculable by a quantum computer. For example, the preparation can refer to transform the scheduling problem into a quantum mechanical description. Moreover, since calculations on the quantum computer are in most cases easier to perform if the variables of the calculated problem refer to binary variables, the preparation can also refer to transforming the scheduling problem or at least parts of the scheduling problem such that the variables of the problem or the part of the problem refer to binary variables. Preferably, the preparation of the scheduling problem comprises determining control operations for controlling the quantum computer to perform the quantum computation of at least a part of the scheduling problem and wherein the scheduling problem preparation unit is further adapted to send the control operations to the quantum computer for performing the quantum computation of the at least a part of the scheduling problem.

Generally, the control operations refer to signals that cause a quantum computer to follow predetermined rules or algorithms to perform a respective calculation indicated by the control operations. In particular, the control operations can refer to signals that merely initialize such a respective calculation but can also refer to signals that determine the complete calculation which is performed by the respective quantum computer. Moreover, the control operations can already be provided in a format that allows for a direct execution of the control operations on the respective quantum computer, but can also be provided in a format that first has to be translated into a format used for controlling the respective quantum computer. For example, if the control operations specify specific manipulations that should be performed on the quantum computer, the signals indicting these manipulations can be translated by a control unit specific to the respective quantum computer into signals interpretable by the quantum computer for performing the operations. In particular with respect to quantum computer, it might be necessary to transform a specific manipulation indicated by the control operations into respective specific control signals for controlling, for instance, a laser unit or an electromagnetic field providing unit to provide laser light or an electromagnetic field, respectively, that allows an intended manipulation of the quantum elements of the respective quantum computer that correspond to the manipulation indicated by the control operations.

In an embodiment, the scheduling problem preparation unit is adapted to prepare the scheduling problem to be solvable on a quantum computer that refers to a quantum annealer or that refers to a gate-based quantum computer. In particular, it is preferred that the quantum computer refers to a quantum annealer.

In an embodiment, the scheduling problem is formulated such that an optimization of the scheduling problem refers to an optimization of the time needed for performing operations indicated by the tasks. Formulating the scheduling problem such that the optimization can refer to an optimization of the time needed for performing operations indicated by the tasks has the advantage that the tasks are performed in as short a time as possible. This allows for a more effective use of the laboratory equipment to analyse and/or test as many probes as possible.

In an embodiment, the apparatus further comprises a constraints determination unit for determining constraints for the scheduling based on the tasks and/or based on laboratory equipment information indicative of technical data of the laboratory equipment, wherein the scheduling problem is formulated further based on the determined constraints. Generally, the constraints represent the restraints and limits provided either by the specific laboratory equipment or by the respective task itself. For example, if a mixer of the laboratory equipment only provides places for five probes for a time slot, it is preferred that the optimization of the scheduling takes this constraint into account, for example, to avoid a scheduling in which in one time slot more than five probes have to be provided to the mixer. Moreover, as already described in detail above, the operations of the tasks have in most cases to be performed in accordance with a fixed predetermined sequence and also in accordance with a fixed predetermined timing to allow for a meaningful result of the analysis or test. Thus, also the tasks themselves can provide respective constraints that are advantageously taken into account during the optimization. The constraints determination unit is then adapted to determine such constraints from the tasks and/or laboratory equipment information. For example, the determination can be based on predetermined rules that allow to derive the constraints from the respective information provided by the tasks and the laboratory equipment information. The identification of the task itself can be associated, for instance, with a predetermined list of constraints or from a list of operations associated with the task the respective constraints can be derived, for instance, by determining the sequence of the respective operations and minimum/maximum times between the operations. Moreover, the laboratory equipment information can directly comprise the respective constraints, for instance, can directly be provided such that the constraints are included into the laboratory equipment information. However, the laboratory equipment information can also refer, for instance, to a list of manufacturer identifications of the laboratory equipment and the constraints determination unit can then be adapted to determine the constraints based on the manufacturer identification of the laboratory equipment, for instance, by looking up technical details of a specific laboratory equipment in a manual provided by the manufacturer of the laboratory equipment. Preferably, the laboratory equipment information is further indicative on whether one or more operations on one or more probes are performable at the same time or sequentially, wherein the constraints are further determined based on this information.

The scheduling problem is then formulated further based on the determined constraints. For example, if the laboratory equipment is not changed for different schedulings, the constraints provided by the laboratory equipment can directly be provided as part of the scheduling problem such that for a new set of tasks only the constraints provided by the task have to be determined and integrated with the scheduling problem already representing the constraints provided by the laboratory equipment. As already described above, for formulating the scheduling problem predetermined rules can be applied that indicate how the tasks and constraints are to be integrated into the scheduling problem, for instance, into a mathematical formation of the scheduling problem. The constraints can be taken into account in the formulation of the scheduling problem as weights or penalties. Higher weights can, for instance, be provided to terms that lead to a fulfilling of the constraints. Penalties on the other hand penalize solutions that do not fulfil the constraints thus increasing the possibility that a solution fulfilling all constraints is provided as final schedule.

In a preferred embodiment, the probe list providing unit is further adapted to provide an updated probe list based on probe data received after the determination of the schedule for the original probe list, wherein the scheduling problem formulation unit is then adapted to reformulate the scheduling problem based on the updated probe list to determine an updated scheduling. In many applications, probes will continuously be provided to the laboratory and then have to be inserted into the scheduling as part of a new probe list. Thus, it is advantageous if in case of a new probe being provided to the laboratory the schedule for the original probe list can be updated. In particular, in this case the original scheduling problem has to be reformulated by the scheduling problem formulation unit to a new scheduling problem that can then again be solved in accordance with the embodiments described above. In this case, it is preferred that the reformulated scheduling problem takes into account a current state of the tasks associated with the original probe list. For example, it is preferred that the probe list providing unit is adapted to continuously update a probe list with respect to a current progress of the schedule, for example, by removing tasks or operations of tasks from the probe list that have already been performed by the laboratory equipment. Thus, if a new probe is added to the probe list, the generated updated probe list can refer to a current state of the schedule of the original probe list and takes this state into account for the further optimization, in particular, for the formulation of the scheduling problem. Generally, since quantum computers allow for a very fast calculation of optimization problems and thus can provide a fast solution also for the updated probe list, the invention as described above allows, in particular, for continuously changing probe lists, a very efficient solution allowing for a full automation of the laboratory equipment.

In a further aspect of the invention, a quantum computer system is presented, wherein the quantum computer system comprises a) an interface unit for receiving, from the apparatus as described above, a scheduling problem formulated such that an optimization of the scheduling problem results in an optimization of a scheduling of tasks associated with a plurality of laboratory probes, wherein the task are to be performed by laboratory equipment on the plurality of laboratory probes, and b) a quantum computer adapted to perform a quantum computation of the received scheduling problem, wherein the interface unit is further adapted to provide a result of a quantum computation of the scheduling problem to the apparatus for further processing.

In a further aspect of the invention, a laboratory system is presented, wherein the laboratory system comprises a) laboratory equipment adapted to perform one or more operations indicated by tasks in an automatic manner on one or more laboratory probes based on control signals, and b) an apparatus as described above, wherein the apparatus provides the determined control signals to the laboratory equipment to perform the determined schedule.

In a further aspect of the invention, a scheduling determination system is presented, wherein the scheduling determination system comprises a) an apparatus as described above, and b) a quantum computer system as described above, wherein the apparatus and the quantum computer system are connected via the interface units.

In a further aspect of the invention, a method for optimizing a laboratory scheduling control is presented, wherein the method comprises a) providing a probe list including probe data for a plurality of probes, wherein the probe data for a probe comprises a probe identification and a task associated with the probe identification indicative of one or more operations to be performed with a predetermined timing by a laboratory equipment on the probe, b) formulating a scheduling problem based on the probe list, wherein the scheduling problem is formulated such that an optimization of the scheduling problem results in an optimization of a scheduling of the tasks associated with the plurality of probes, c) sending, via an interface, the scheduling problem to the quantum computer to be optimized utilizing quantum computing and receiving, via the interface, a result of the optimization of the scheduling problem from the quantum computer, d) determining a schedule for the probe list based on the received result of the quantum computation, and e) determining a control signal for controlling the laboratory equipment based on the determined schedule.

In a further aspect of the invention, a computer program product for optimizing a laboratory scheduling control is presented, wherein the computer program product comprises program code means for causing the apparatus as described above to execute the method as described above.

In a further aspect of the invention control signals are presented for controlling a laboratory equipment based on a determined schedule, wherein the control signals are generated utilizing an apparatus or method as described above.

In a further aspect of the invention a use of the apparatus or method as described above is presented for controlling a laboratory equipment based on a determined schedule.

In a further aspect of the invention, a scheduling problem preparation apparatus is presented, wherein the scheduling problem preparation apparatus comprises a) an interface for receiving a scheduling problem, wherein the scheduling problem is formulated such that an optimization of the scheduling problem results in an optimization of a scheduling of tasks associated with a plurality of probes, b) a processor configured for preparing at least a part of the scheduling problem such that the optimization of the scheduling problem is performable utilizing a quantum computation, and c) an interface for providing the prepared at least a part of the scheduling problem to a quantum computer for calculating the prepared at least a part of the scheduling problem.

It shall be understood that the apparatus as described above, the methods as described above, the apparatuses as described above and the computer program product as described above have similar and/or identical preferred embodiments, in particular, as defined in the dependent claims.

It shall be understood that a preferred embodiment of the present invention can also be any combination of the dependent claims or above embodiments with the respective independent claim.

These and other aspects of the present invention will be apparent from and elucidated with reference to the embodiments described hereinafter.

In the following drawings:

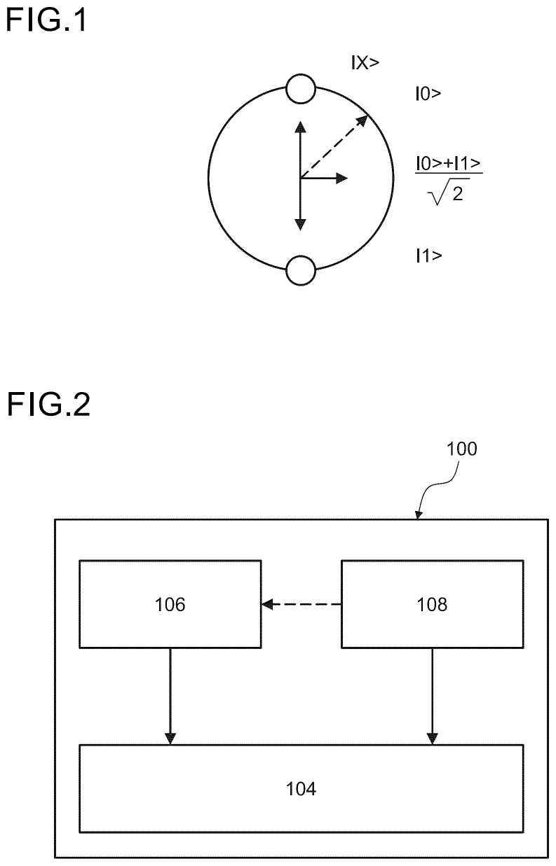

FIG. 1 illustrates a state representation of a qubit as used in a quantum computing device,

FIG. 2 illustrates a schematic example of a quantum computing device with qubits as calculation unit,

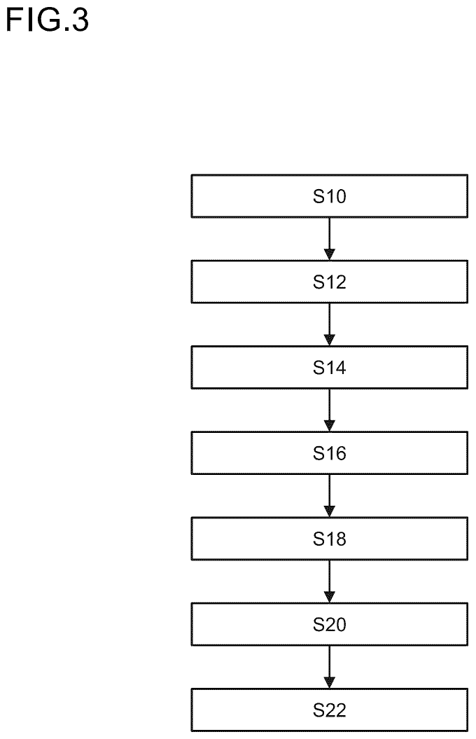

FIG. 3 illustrates a schematic example method for generating a control signal to perform operations on a quantum computing device and for processing measurement signals from the quantum computing device,

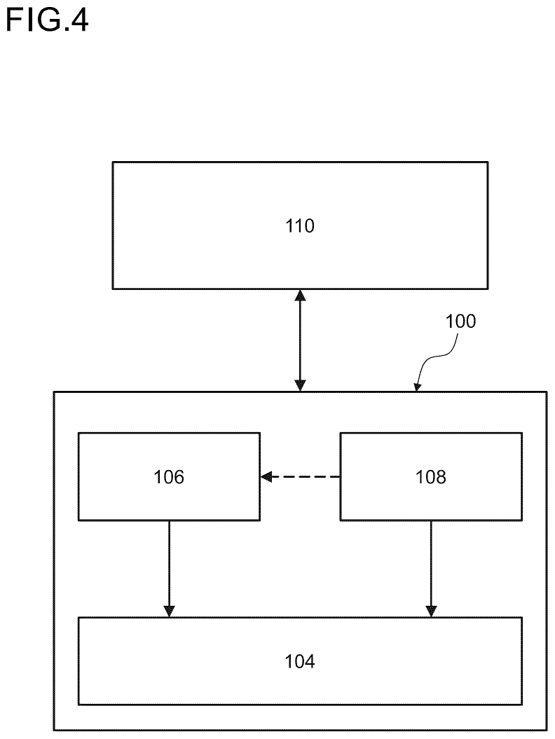

FIG. 4 illustrates a schematic example of a hybrid system including a classical and a quantum computing device,

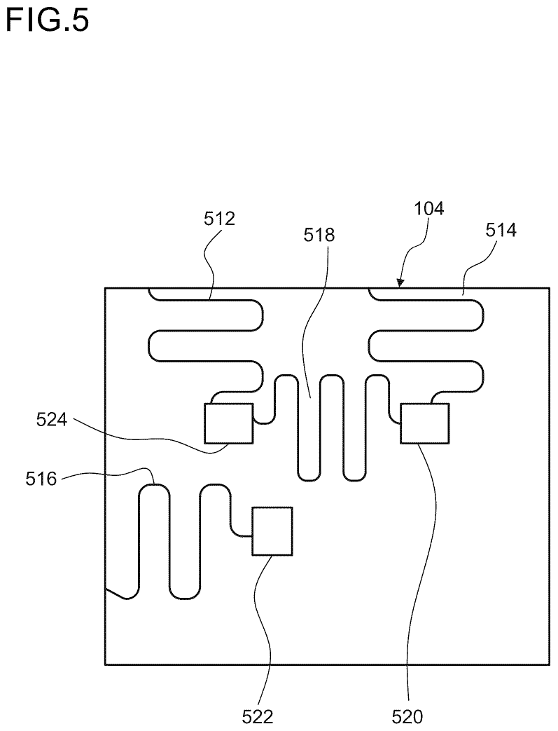

FIG. 5 illustrates a schematic example of a quantum computing device based on superconductors,

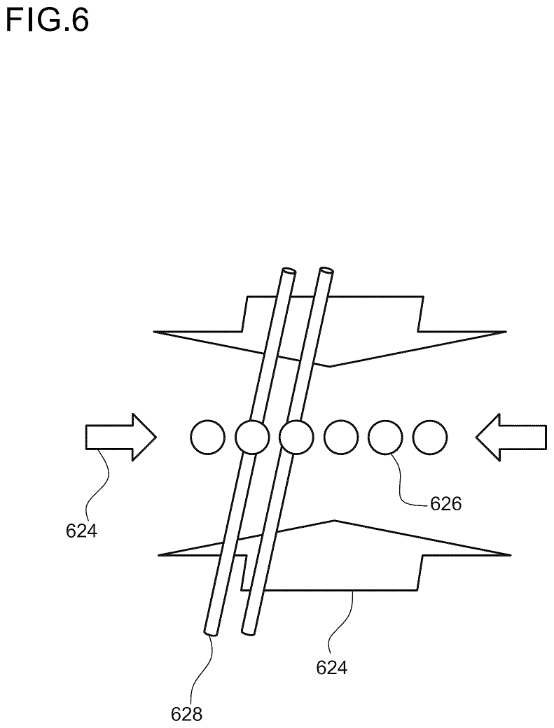

FIG. 6 illustrates a schematic example of a quantum computing device based on trapped ions,

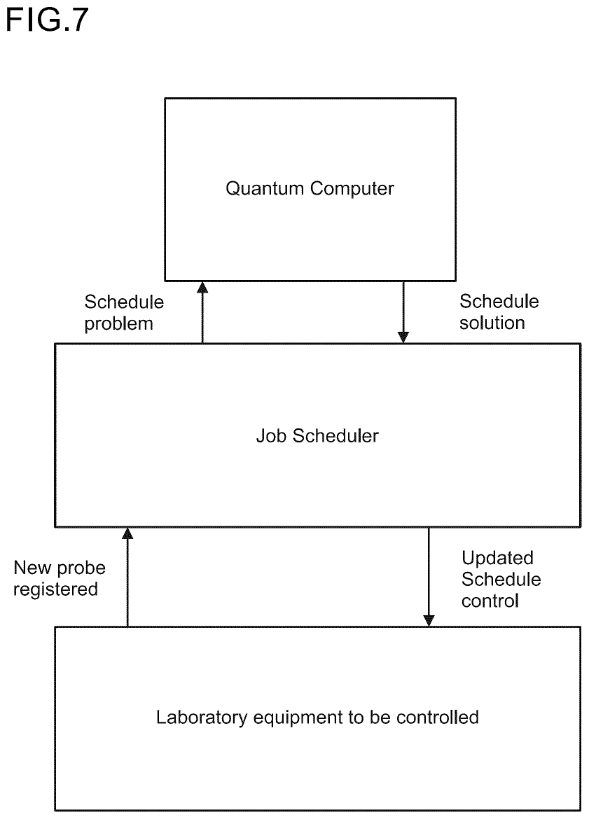

FIG. 7 shows schematically and exemplarily a general structure of a scheduling system for optimizing a laboratory scheduling control,

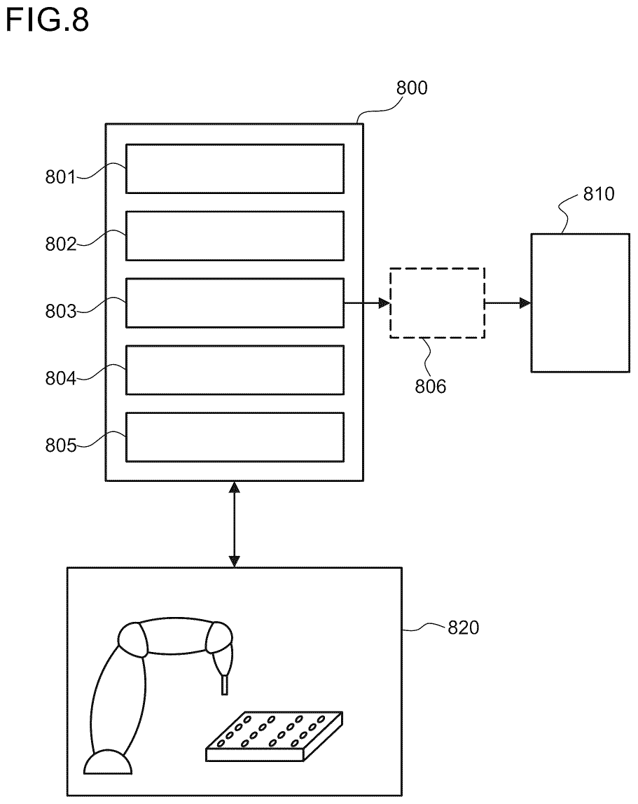

FIG. 8 shows schematically and exemplarily an embodiment of the apparatus 800 for optimizing a laboratory scheduling control,

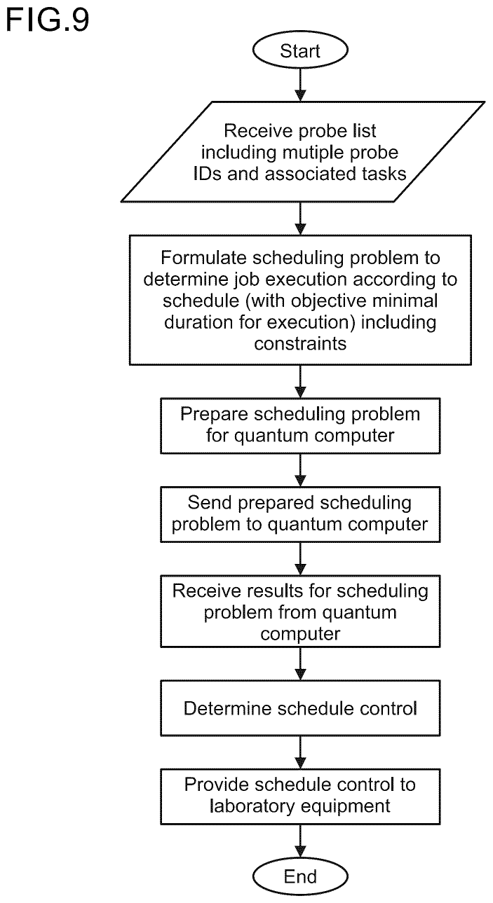

FIG. 9 shows schematically and exemplarily a flowchart of a method for optimizing a laboratory scheduling control,

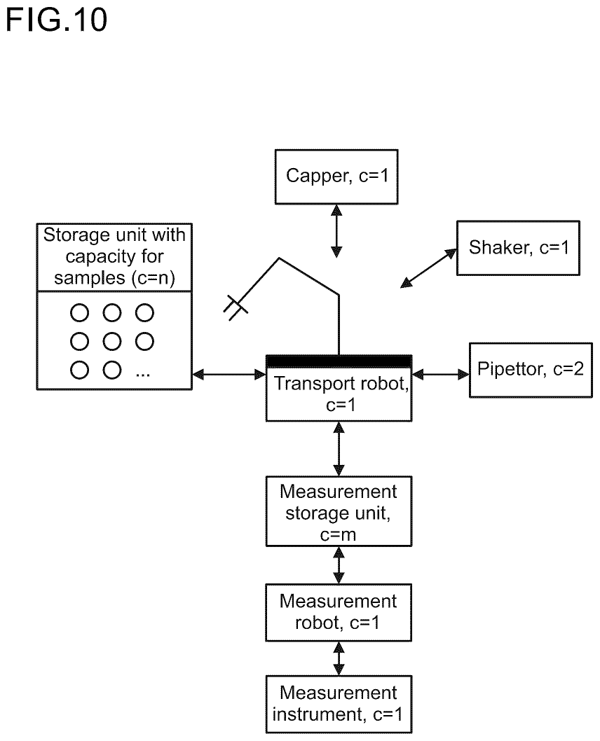

FIG. 10 shows schematically and exemplarily a high throughput facility to which the invention can be applied,

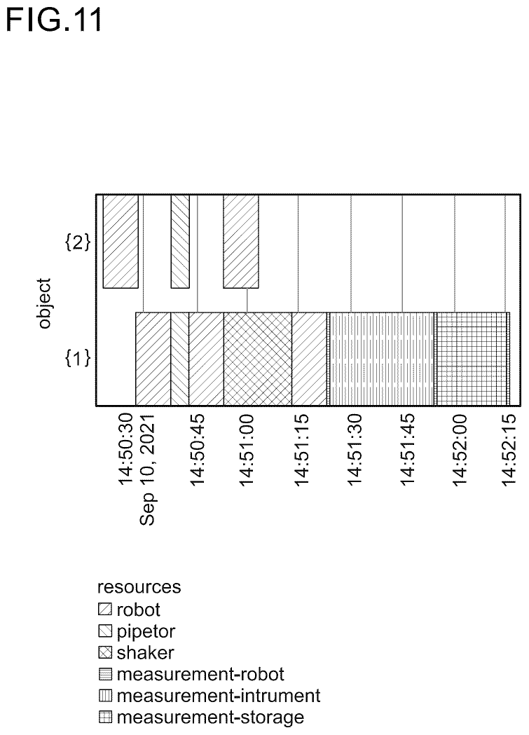

FIG. 11 shows a schematic example of a determined schedule for two probes, and

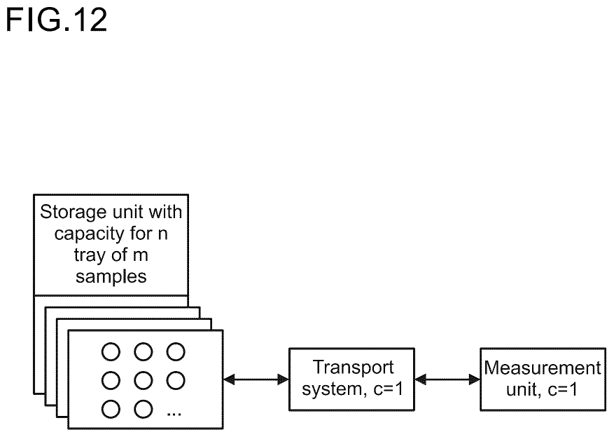

FIG. 12 shows schematically and exemplarily a long storage facility to which the invention can be applied.

### DETAILED DESCRIPTION OF DRAWINGS

In the following first a short introduction into the general basic principles of quantum computers and the performance of calculations of quantum computers will be provided. Further, general principles can also be found in “Quantum Computation and Quantum Information: 10th Anniversary Edition”, M. A. Nielsen and I. L. Chuang (2010).

Classical computing devices use processors which are based on transistors. The state of each transistor has two controllable states 1 or 0 representing a digital binary or a bit. To perform operations on a classical computing device a human readable program code is translated via a compiler into machine-readable instructions. Machine-readable instructions are control signals, e.g. voltage settings, for each transistor. Representations of the machine-readable instructions may include binary or hexadecimal representations. Based on such machine-readable instructions, the operations are performed on the processor of a classical computing device.

Quantum computation is a relatively new computation method that uses quantum effects, such as superposition and entanglement, to perform certain computations more efficiently than classical digital computers. In contrast to digital computers, which represent information in the form of bits (e.g., “1” or “0”), as described above, quantum computing devices, i.e. quantum computers, use qubits, i.e. quantum bits, to represent information. Quantum computing devices are based on quantum elements adhering to the physics of quantum mechanics, such as superconductors, ions, atoms, quantum dots, photons, particle spins, bosons or the like. These quantum elements may be manipulated in a controlled manner to perform operations.

Although qubits and their manipulation may be described in terms of their mathematical properties, each such qubit may be implemented in a physical quantum element in any of a variety of different ways. Examples of such quantum elements include superconducting materials, trapped ions, photons, optical cavities, individual electrons trapped within quantum dots, point defects in solids (e.g., phosphorus donors in silicon or nitrogen-vacancy centers in diamond), molecules (e.g., alanine, vanadium complexes), or any medium that exhibits qubit behavior comprising quantum states and transitions there between that can be controllably induced or detected.

Generally, for any given physical quantum element that implements a qubit, any of a variety of properties of that physical unit may be chosen to implement the qubit. For example, if electrons are chosen to implement qubits, then the x, y or z component of an electron spin degree of freedom can be chosen as the property of such electrons to represent the states of such qubits. For any particular degree of freedom, the physical quantum elements can be controllably put in a state of superposition or entanglement and measurements can then be taken in the chosen degree of freedom to obtain readouts of qubit values.

In contrast to transistors of classical computing devices each quantum element of quantum computing devices can not only take the basis states |1> or |0> but also any superposition of such basis states, such as state |X>. The state of each quantum element is represented by a state of a quantum bit, i.e. qubit, as illustrated in the two-dimensional simplification of FIG. 1. To represent such states Dirac notation is commonly used in quantum mechanics. In Dirac notation a state in a n dimensional, complex vector space, such as a Hilbert space, is represented in bracket notation, for example |X>. According to conventional terminology, the superposition of “0” and “1” states in a quantum computing device can be represented as α|0>+β|1>. The states “0” and “1” or bits of the classical computing device are similar to the basis states |0> and |1> or quantum bits of the quantum computing device, respectively. The value |α|² represents the probability that the qubit will be measured in the |0> state, while the value |β|² represents the probability that the qubit will be measured in the |1> state. If more than one qubit is present, two or more qubits may be entangled. Entanglement means that the state of one qubit is dependent on the state of at least one other qubit and vice versa, wherein further in the entangled state the respective qubits cannot be regarded as individual qubits anymore. Generally, a register of N qubits in a quantum computer can be put into a superposition of basis states at once whereas a register of N classical bits can only be in a single basis state at once. Thus, in contrast to classical computing devices on a quantum computing device 2^{N} basis states can be manipulated and processed simultaneously allowing for exponential intrinsic parallelism.

To perform operations on the quantum computing device the computational method to solve a given problem may be translated into qubit manipulations, which may be translated into control signals for manipulating qubits. Representations of the machine-readable instructions may include common quantum mechanical representations of operations in the Hilbert space. Depending on a specific realization of the quantum computer different representations of the qubit states may be chosen. Any state preparation on the quantum computing device may be represented by a manipulation acting on the qubit states. A manipulation may be translated into control signals to control a respective part of the quantum computer, which depend on the type of quantum computing device used. This way based on the manipulation acting on the qubit states, operations may be performed on the quantum equivalent of a classical processor as part of the quantum computing device.

In gate-based quantum computer systems the manipulations acting on the qubit states may generally be one- or multi-qubit operations. A one-qubit operation may change the state of one qubit e.g., into a specific superposition which corresponds to a rotation of the vector |X> as illustrated in FIG. 1. For example, in a superconducting quantum computer this can be accomplished by microwave pulses or in a trapped-ion quantum computer by irradiation of the ion with a laser beam. A multi-qubit operation may create entanglement between two or more qubits. For example, in a superconducting quantum computer this may be achieved by connecting qubits via an intermediate electrical coupling circuit or in a trapped-ion quantum computer via controlling the collective vibrations of the trapped ions.

Generally, to prepare manipulations for solving a given problem on a quantum computer a respective quantum mechanical representation of the problem may be translated into qubit manipulations, which are carried out to prepare a solution of the given problem. After the preparation of the predetermined solution, i.e. after the application of the operations to the qubits of the quantum computer, a projective measurement of all individual qubits is carried out returning either 0 or 1 for each qubit. On the quantum computing device this measurement is achieved by applying a hardware-specific readout protocol of a series of readout manipulations including, for example, in the case of gate-based quantum computers, control pulses and monitoring the response to control pulses. For example, a superconducting qubit may be coupled to a hardware resonator. The measured shift of the resonator frequency allows to determine the state of the qubit as this shift depends on the state of the coupled qubit. In case of trapped ions, for example, an optical readout may be used, e.g. the state of the qubit is 1 if the ion emits light or 0 if the ion does not emit light or vice versa. This way qubits may be used, in particular, on gate-based quantum computers, to implement logical circuits or gates as in classical computing devices.

In FIG. 2 a schematic example of a quantum computer is illustrated. The quantum computing device 100 shown in FIG. 2 includes a quantum register 104 configured to perform the quantum computation, a manipulation part 106 configured to manipulate the quantum register, in particular, quantum elements forming the qubits, and a readout part 108 configured to collect measurement signals from the quantum register 104 for reading out the qubits after a quantum mechanical calculation. The manipulation part 106, in particular, provides manipulation signals for manipulating the quantum register, wherein the manipulation signals are generated based on received control signals that are determined based on the respective operations that should be performed on the qubits. In some embodiment a feedback loop between the manipulation part 106 and measurement part 108 can be provided. In contrast to classical computing, where one measurement cycle provides the state of a transistor, quantum computing includes performing multiple measurement cycles to provide a probability density or a probability for the qubit states in case of gate-based quantum computers or to determine the measurement with the lowest energy in case of a quantum annealer.

The quantum register 104 can be based on different quantum elements representing the qubits. In some embodiments of gate-based quantum computers the qubits may be implemented by photons as quantum elements. Such optical quantum computing devices may include lasers that generate photons that are provided to a waveguide. A beam splitter can be provided for manipulating the photon states based on manipulation signals such as a mechanical rotation applied to a mirror. The measurement part 108 can in such an embodiment be a photon detector, and the measurement signals can be photons.

In other embodiments of gate-based quantum computers the qubits can be implemented by electronic states of ions trapped in a magnetic field. The manipulation part 106 can in such a case utilize a laser, and the manipulation signals can cause the providing of control laser pulses. Moreover, in this case, the readout part 108 can be a photon detector combined with read-out laser pulses, and the measurement signals 102 may be photons. Other qubit implementations may be based on superconductors as quantum elements, semiconducting material with anyons as quantum elements, or the like.

FIG. 3 illustrates a schematic exemplary method for generating a control signal to perform manipulations on a quantum computer and for processing measurement signals from the quantum computing device. In most embodiments of quantum computing devices known to date, the control signals for the quantum computing device are prepared on a classical computing device and the measurement signals provided by the quantum computing device are further processed on the classical computing device. Other embodiments are, however, conceivable as quantum computing devices mature. In the following example, the quantum computer refers to a gate-based quantum computer for which the manipulations refer to operations on the quantum elements of the quantum computer.

For generating the control signal to perform operations on the quantum computing device, the problem to be solved with the aid of the quantum computing device is provided in step S10, preferably, in a mathematical description. Such problem may for instance include determining a material property based on the mathematical description of the material's electronic structure. Other problems may include optimization problems and associated objective functions. Based on the problem to be solved, an operation description of the problem or a sub-problem may be generated in step S12, wherein the operation description comprises the operations to be applied to the qubits of the quantum computer to solve the problem in the quantum mechanical calculation. Further, the operation description can include a reference state that allows to generate a representation of an initial qubit state on the quantum computer on which the further operations are then applied by manipulating the qubit states. Based on the operation description control signals can then be generated in step S14 to control the quantum computer, for instance, by providing the control signals to the manipulation unit that can then manipulate the qubit states based on the control signals. In step S16 the manipulation unit then applies the manipulation operations to individual or multiple qubits of the quantum computer, wherein based on the manipulation operations the qubits perform the quantum mechanical calculation. After the manipulation, measurement signals can be generated to determine the result of the quantum mechanical calculation in step S18. This step can include a read-out, i.e. measurement, of the qubit states after applying the manipulation operations to the initial qubit states. The measurement signals can in step S20 then be translated into a measured quantity on the classical computer and in case of a sub-problem fed back into the problem to be solved. Finally, the result of the problem calculation including the quantum mechanical calculation can be provided on the classical computing device in step S22.

FIG. 4 illustrates a schematic example of a hybrid system including a classical and a quantum computing device. As described with respect to the method illustrated in FIG. 3, quantum computing devices are often used in connection with classical computing devices. As shown in FIG. 4 a problem preparation system can be realized as a classical computing device 110 performing, for instance, steps S10, S12, S20, S22 of the method illustrated in FIG. 3. A controlling unit can then be provided as interface between the classical computing device 110 and the quantum computer 100, wherein the controlling unit can also be a classical computing device, for instance, performing step S14. The control unit can then be communicatively coupled with the manipulation part 106 that can control the manipulators of the quantum computing device. Also, the manipulation part 106 can be realized as a classical computing device, for instance, a classical controlling hardware for the control of specific hardware components of the quantum computer that perform the manipulation of the qubits. However, the manipulation part 106 is generally regarded as part of the quantum computer, since it directly influences the quantum register. The quantum computing device 100 is adapted to perform the quantum operations in step S16, in particular, by the manipulation of the qubits of the quantum register. The measurement part 108 that is also generally regarded as part of the quantum computing device can then perform the step S18 by utilizing classical hardware. The measurement part 108 can then be communicatively coupled to the preparation system 110 for further processing of the measurement signals.

FIG. 5 illustrates a schematic example of a quantum computing device based on superconductors. Superconducting quantum computing devices are one of the solid-state quantum computing technologies. Here the quantum register 104 can include superconducting circuits 520, 522, 524 based on Josephson junctions. The qubits can then, for instance, refer to charge, flux, transmon, or phase qubits depending on the quantity of the superconducting circuits that are chosen to represent the qubits. FIG. 5 refers to a simplified illustration of a superconducting quantum computer utilizing charge qubits. For charge qubits the different states of the qubit are represented by an integer number of Cooper pairs on a superconducting island. In case of gate-based quantum computing quantum manipulations can then be performed by manipulating the qubits through microwave pulses. Resonators 512, 514, 516 can be utilized to manipulate the state of the qubits by applying the microwaves or for reading out the state of the qubits by measuring respective microwaves, wherein generally different resonators are used for the manipulation of the state of the qubits and the readout of the qubits. Moreover, resonator 518 can be utilized for applying microwaves that entangle the qubits. However, instead of resonator 518 the entanglement can also be achieved by an inductive or capacitive coupling of the superconducting circuits or even by providing another qubit, here a superconducting circuit, between the two be entangled qubits.

On an operational level such systems are maintained at extremely low temperatures, e.g., in the tens of mK. The extreme cooling of the systems keeps superconducting materials below their critical temperature and helps to avoid unwanted state transitions. To maintain such low temperatures, the quantum information processing systems may be operated within a cryostat, such as a dilution refrigerator. In some implementations, control signals are generated in higher-temperature environments, and are transmitted to the quantum computer using shielded impedance-controlled GHz capable transmission lines, such as coaxial cables. In some implementations, the state measurement of superconducting qubits is achieved using a dispersive detection scheme. In order to read out or detect the state of any qubit, a probing signal, e.g., a travelling microwave, may be excited along a readout transmission line coupled to the qubit via a respective readout resonator. The frequency of the probing signal can be in the vicinity of the resonance frequency of the readout resonator. Depending on the internal quantum mechanical state of the qubit, the intensity or phase of the probing signal transmitted along the readout transmission line may be altered because the reflectivity of the readout resonator coupled to the qubit changes depending on the state of the qubit. This allows for the state detection of the qubits, wherein during the readout of a qubit state the state of the qubit collapses, i.e. is projected with the respective probability onto one of the basis states. By performing the quantum mechanical calculation and the readout a plurality of times, for example, respective probabilities can be determined. Further details for superconducting quantum devices are described e.g. in documents EP 3830867 A1, EP 3449427 A1, U.S. 2020272925 A1, CN 212061223 U and U.S. 2019019099 A1.

FIG. 6 illustrates a schematic example of a quantum computing device based on ions in an ion trap. Similar to neutral atom traps ion traps with, e.g. positively charged Calcium ions, can be used to implement the quantum computing device. Here ions 626 are trapped in an oscillating electromagnetic field 624 inside a high or ultra-high vacuum. The ions 626 are laser cooled and held in the oscillating electrical field 624. For qubit manipulation such as superposition or entanglement laser light 628 at different frequencies may be used.

Generally, based on the above described quantum computer realizations gate-based type calculations can be performed on a quantum computer hardware architecture. The gate-based type calculation is based on quantum gates. In contrast to classical gates, there is an infinite number of possible single-qubit quantum gates that can change the state vector of a qubit. Changing the state of a qubit state vector typically is referred to as a single qubit rotation, and may also be referred to herein as a state change or a single-qubit quantum gate operation. A rotation, state change, or single-qubit quantum gate operation can be represented mathematically by a unitary 2×2 matrix with complex elements. A rotation corresponds to a rotation of a qubit state within its Hilbert space, which can be conceptualized as a rotation of a vector on the Bloch sphere, wherein the Bloch sphere is generally known as a geometrical representation of the space of the pure states of a qubit. Multi-qubit gates alter the quantum state of a set of qubits. For example, two-qubit gates rotate the state of two qubits as a rotation in the four-dimensional Hilbert space of the two qubits, wherein, as generally known, the Hilbert space is an abstract vector space possessing the structure of an inner product that allows length and angle to be measured. Furthermore, Hilbert spaces are complete, i.e. there are enough limits in the space to allow the techniques of calculus to be used.

In the following the term operation description refers to a representation of a problem that comprises either a sequence of quantum operations that should be applied during a quantum mechanical calculation of the problem on a gate-based quantum computer or to the manipulations performed on the quantum elements during a quantum mechanical calculation of the problem on a quantum annealer. The term “quantum operation” can include in the context of this invention all types of quantum gates as described above and all manipulations known for quantum annealers as will be described in more detail below. Further, in some applications the quantum operations can also include measurement manipulations. This allows to implement algorithms using a measurement feedback. For example, in such an algorithm a quantum computer can execute the quantum gates defined by the sequence of quantum operations and then measure only a subset, i.e., fewer than all, of the qubits or other calculation elements, like the bosonic field states, in the quantum computer, and then decide which further quantum operations to execute next based on the outcome of the one or more measurements. In particular, measurement feedback can be useful for performing quantum error correction, but is not limited to use in performing quantum error correction.

Not all quantum computers are gate-based quantum computers. Embodiments of the present invention are not limited to utilizing gate-based quantum computers. As an alternative example, embodiments of the present invention can also utilize, in whole or in part, a quantum computer that is implemented using a quantum annealing paradigm which is an alternative to the gate-based quantum computing paradigm. More specifically, quantum annealing is a metaheuristic for finding the global minimum of a given objective function over a given set of candidate solutions (candidate states), by a process using quantum fluctuations. In particular, quantum annealing is closely related to adiabatic quantum computing.

Generally, also quantum annealing procedures start with utilizing a classical computer providing or generating an initial Hamiltonian and a final Hamiltonian based on a computational problem to be solved, and providing the initial Hamiltonian, the final Hamiltonian and an annealing schedule as input to a quantum computer. In case of an annealing procedure in which an optimization problem is solved, preferably, the Hamiltonian refers to a Ising Hamiltonian representing the optimization problem, wherein the final Hamiltonian refers in this case to or a good approximation of the ground state of the Ising Hamiltonian. The quantum computer is then adapted, for instance, by utilizing a respective control unit controlling a manipulation part of the quantum computer, to prepare a relatively easy to prepare initial state, such as a quantum-mechanical superposition of all possible states, e.g. candidate states, with equal weights, based on the initial Hamiltonian. After the preparation of the initial state on the quantum computer, the initial state is then evolved according to the annealing schedule following a time-dependent Schroedinger equation referring to a natural quantum-mechanical evolution of the physical system of the quantum computer. More specifically, the state of the quantum computer undergoes time evolution under a time-dependent Hamiltonian, which starts from the initial Hamiltonian and terminates at the final Hamiltonian. If the evolution rate is slow enough, the system stays close to the ground state of the instantaneous Hamiltonian. At the end of the time evolution, the set of qubits, i.e. quantum elements, on the quantum annealer is in a final state, which is expected to be close to the ground state of the Ising Hamiltonian that corresponds to a solution to the original problem, referring, for instance, to an optimization problem. The final state of the quantum computer can then be measured, thereby producing results that can be utilized for solving the original problem. The measurement operation can be performed, for example, in any of the ways described already above. A classical computer can then perform postprocessing on the measurement results to produce an output representing a solution to the original computational problem. A quantum annealer as described above can, for instance, be realized on a superconducting quantum computer hardware.

Moreover, embodiments of the present invention can also utilize, in whole or in part, quantum computers that are implemented using a one-way quantum computing architecture, also referred to as a measurement-based quantum computing architecture. More specifically, the one-way or measurement-based quantum computer refers to a method of quantum computing that first prepares an entangled resource state, usually a cluster state or graph state, then performs single qubit measurements on it. It is “one-way” because the resource state is destroyed by the measurements. In such an architecture, the outcome of each individual measurement is random, but they are related in such a way that the computation always succeeds. In general, the choices of basis for later measurements need to depend on the results of earlier measurements, and hence the measurements cannot all be performed at the same time.

FIG. 7 shows schematically and exemplarily a general structure of a scheduling system for optimizing a laboratory scheduling control. A more detailed embodiment is shown, for instance, in FIG. 8. FIG. 7 illustrates schematically the principle structure and relations between the quantum computer, a job scheduler referring to or comprising, for instance, the apparatus for optimizing a laboratory scheduling control as will be described in detail with respect to FIG. 8, and the laboratory equipment. In particular, the job scheduler is configured to formulate a schedule problem, for instance, based on a probe list, to provide at least a part of the schedule problem to the quantum computer for calculation. The schedule solution, in particular, the results of the quantum computer calculation indicative for the schedule solution, are then again provided to the job scheduler such that the job scheduler can determine based on the results of the quantum computer calculation a job schedule and provide respective schedule control signals to a laboratory equipment to be controlled. In embodiments in which new probes can be added to the probe list also during the performance of the previously determined schedule for the original probe list, the laboratory equipment can be adapted to register such a new probe. For example, a barcode scanner of the laboratory equipment can notice a probe with a new identifier indicated by the barcode and can then provide the newly registered probe together with further information, for instance, a task associated with the new probe to the job scheduler. The job scheduler can then reformulate the scheduling problem and again, while utilizing the quantum computer, determine an updated schedule control that is provided to the laboratory equipment.

FIG. 8 shows schematically and exemplarily an embodiment of the apparatus 800 for optimizing a laboratory scheduling control in more detail. In particular, it is preferred that the job scheduler as illustrated in FIG. 7 comprises an apparatus 800. The apparatus 800 comprises a probe list providing unit 801, a scheduling problem formulation unit 802, a quantum computer interface unit 803, a schedule determination unit 804 and a control signal determination unit 805. Optionally, the apparatus 800 can further comprise a scheduling problem preparation unit 806 as part of the quantum computer interface unit 803. However, in other embodiments the scheduling problem preparation unit 806 can also be a standalone system or can be completely omitted. Generally, the apparatus can be provided in any form of hardware and/or software. In particular, the apparatus 800 can comprise one or more processors that are configured to provide the functions defined by the units of the apparatus 800.

The probe list providing unit 801 is configured to provide a probe list including probe data for a plurality of probes. For example, the probe list can be generated by the laboratory equipment 820 by reading out information from identification systems provided with the probes, for instance, inserted into a probe tray. For example, the probes inserted into the probe tray can comprise a barcode, QR code or RFID chip providing when read out not only an identification of the probe but, for example, also an indication of the task that should be performed on the probe. However, the probe list can also be provided, via an input unit, by a user inputting the probe list. In particular, the probe list includes probe data of a plurality of probes, wherein the probe data comprises a probe identification, for instance, a probe identification number, and a task associated with the probe identification. The task is generally indicative of one or more operations to be performed in a predetermined timing by the laboratory equipment 820 on the respective probe. For example, the task can indicate that a respective probe identified by a respective identification number shall undergo a PCR testing procedure. Such a PCR testing procedure as task indicates a plurality of operations to be performed on the probe, like operations to add a chemical to the probe, to deplete a probe, to heat a probe, to mix a probe, or to measure one or more characteristics of the probe. For each of these operations, one or more of the laboratory equipment can be automatically utilized if provided with the respective control signals.

The scheduling problem formulation unit 802 is then adapted to formulate a scheduling problem based on the probe list. The scheduling problem is formulated such that an optimization of the scheduling problem results in an optimization of a scheduling of the tasks associated with the plurality of probes. For example, the scheduling problem formulation unit 802 can be adapted to utilize predetermined rules to formulate the scheduling problem, preferably, in a mathematical formulation. However, the scheduling problem formulation unit 802 can also be adapted to formulate the scheduling problem in a computer guided user interaction procedure, in which the scheduling problem formulation unit 802 can, for instance, provide a user with a plurality of possibilities of formulating a scheduling problem based on the problem list and then allow the user to select one or more parts of the scheduling problem to be utilized. The scheduling formulation unit 802 can be adapted to formulate the scheduling problem, for example, according to the following rules. First all dependency constraints are added to the optimization problem formulation, wherein the dependency constraints are implied by the individual tasks associated with the probes and refer to dependencies between the operations of a task. This can include, for example, just an ordering corresponding to the ordering of the operations of a task but also a particular waiting time between operations or tasks. In a next step known capacity constraints can be added to the optimization problem formulation, wherein the capacity constraints refer to known limited capacities of the equipment utilized for a task, e.g., a robot can only transport one object at a time. In the last step the optimization problem formulation is formulated such that an optimization of the optimization problem formulation referring to the scheduling problem minimizes the time needed to perform all tasks of the probe list. Additional details and principles for formulating optimization problems based on general task lists that can also be applied and, for instance, utilized as rules by the scheduling problem formulation unit, can be found, for instance, in the following documents. In the patent application EP 3 859 470 A1, incorporated by reference, for example, an objective problem is formulated to find an optimal combination of components forming a product, wherein the objective function is formulated as quadratic unconstrained binary optimization based on a variance between features of possible combinations of components and a target feature. In the article “Job Shop Scheduling Solver based on Quantum Annealing” by D. Venturelli et al., Proc. of ICAPS-16 Workshop on Constraint Satisfaction Techniques for Planning and Scheduling (COPLAS), pages 25 to 34 (2016), incorporated by reference, a job-shop scheduling problem to be solved on a quantum annealer is formulated by utilizing a time-indexed quadratic unconstrained binary optimization. Also in the article “Traffic Flow Optimization Using a Quantum Annealer” by F. Neukart et al., Frontiers in ICT, vol. 4, page 29 (2017), incorporated by reference, a quadratic unconstrained binary optimization is utilized for formulating a traffic flow problem to be solved on a quantum annealer.

The quantum computer interface unit 803 is configured to provide an interface for interfacing with the quantum computer 810 for utilizing the quantum computer for calculating the scheduling problem. Moreover, the quantum computer interface unit 803 can also be configured to receive the result of the quantum computation of the scheduling problem from the quantum computer 810, wherein the result is indicative of the optimized scheduling problem. Generally, the quantum computing interface unit 803 can directly be configured for controlling the quantum computer 810 in order to perform calculations of at least a part of the scheduling problem. For example, the quantum computer interface unit 803 can provide control operations to the quantum computer that directly control the manipulations performed on the quantum elements by the quantum computer 810. However, alternatively, a scheduling problem preparation unit 806 can be provided either as part of the quantum computer interface unit 803 or as a standalone system that allows for the preparation of the scheduling problem. Generally, algorithms and methods for calculating optimization problems on a quantum computer are already known.

Examples for such algorithms, processes and methods for calculating optimization problems on a quantum computer that can also be applied to the scheduling problem can be found, for instance, in the following articles. In the article “Quantum computing based hybrid solution strategies for large-scale discrete-continuous optimization problems” by A. Ajagekar et al., Computers & Chemical Engineering, vol. 132, page 106630 (2020), incorporated by reference, a mixed-integer linear programming model is utilized to formulate a job-shop scheduling problem and a hybrid solution strategy that integrates a decomposition-based algorithm with a Quantum computer solution technique for the global optimization of this job-shop scheduling problem is utilized. The article, “Digital Annealer for High-Speed Solving of Combinatorial Optimization Problems and Its Applications” by S. Matsubara et al., 25th Asia and South Pacific Design Automation Conference (ASP-DAC), pages 667 to 672 (2020), incorporated by reference, utilizes a digital annealer to solve combinatorial optimization problems based on an Ising formulation of an energy function. and “Quantum algorithms for process parallel flexible job shop scheduling” by B. Denkena et al., CIRP Journal of Manufacturing Science and Technology, vol. 33, pages 100 to 114 (2021), incorporated by reference, describes a detailed algorithm for processing job-shop scheduling problems formulated as quadratic unconstrained optimization problem by utilizing quantum annealers. The such prepared scheduling problem or at least a part of the scheduling problem is then calculated on the quantum computer 810 and the result provided via the quantum computer interface unit 803 back to the apparatus 800.

The schedule determination unit 804 is then adapted to determine a schedule for the probe list based on the received result of the quantum computation. For example, if the result of the quantum computation refers to a calculation of only a part of the scheduling problem, the schedule determination unit 804 can be adapted to derive from the result of the quantum computation the optimized scheduling problem and thus the solution of the scheduling problem. However, if the result of the quantum computation is directly indicative of the solution of the scheduling problem, the schedule determination unit 804 can be adapted to transfer the information provided by the solution of the scheduling problem into a format of the schedule that can be directly utilized, for instance, by a control signal determination unit 805 that can be realized as a laboratory management unit to provide the respective control signals for controlling the laboratory equipment 820. For example, the schedule determination unit 804 can determine based on the result of the quantum computation, in particular, based on the result of the optimized scheduling problem, which laboratory equipment has to perform which task on which probe at which time slot. Based on this determined schedule, the control signal determination unit 805 is then configured to determine a control signal for controlling the laboratory equipment 810 in accordance with the determined schedule. For example, the control signal determination unit 805 can be communicatively coupled or can refer to an automated laboratory management system that allows for a direct control of the laboratory equipment 820, for instance, via wireless or wired signals. Based on the respective control signals, the laboratory equipment 820 then performs the schedule for all probes of the probe list.

FIG. 9 shows schematically and exemplarily a flowchart of a method for optimizing a laboratory scheduling control. In particular, the method starts with receiving a probe list including multiple probe identifications and the associated tasks. In a next step, based on the received probe list, a scheduling problem is formulated to determine a schedule for the job execution, i.e. for executing the operations of the associated tasks on the probes, such that the duration of the execution time becomes objectively minimal. Preferably, the formulation of the scheduling problem takes all known constraints into account. Constraints can in particular refer to physical constraints of the laboratory equipment, like the movements possible for the laboratory equipment, the possible number of probes that can be processed by a laboratory equipment in parallel or sequentially, possible fluid amounts that can be processed by the laboratory equipment, etc. Moreover, the constraints can refer to constraints provided by the tasks themselves, for instance, by the operations that have to be performed by the laboratory equipment to execute the tasks. For example, a task can require that the operations are performed in a predetermined sequence and also with respect to a predetermined timing. Thus, taking these constraints into account does not only lead to a better solution but also allows to avoid solutions that cannot be realized by the laboratory equipment. In a next step, problem is prepared to be calculated at least partly on a quantum computer. For example, the preparation can refer to transforming the scheduling problem into a formulation that is based on binary variables that are easier to calculate on quantum computers. In the next step, the prepared scheduling problem is then sent to the quantum computer for calculation. After the calculation of the prepared scheduling problem on the quantum computer, the results of the quantum computation are received and in a further step a solution of the scheduling problem is determined by determining a schedule control for controlling the laboratory equipment. In the last step, the schedule control is then provided to the laboratory equipment in order to perform the schedule on the probes of the probe list.

In the following, some further details of preferred embodiments of the invention will be described. In a preferred application of the invention the laboratory equipment refers to a high throughput equipment utilized in a high throughput facility. A schematic and exemplary embodiment of such equipment is illustrated in FIG. 10. FIG. 10 shows schematically possible equipment utilized in a high throughput process, in particular, a transport robot configured to transport probe containers from one processing station to another processing station, a storage unit for storing probe container, a capper, a shaker, a pipettor, a measurement storage unit, a measurement robot and a measurement instrument. Each of these units is associated with a processing station at which an operation of a task can be performed on a probe. Further, FIG. 10 provides information on a capacity of each unit, for example, the transport robot has a capacity of 1 probe container meaning that the robot can transport one probe container at a time. A result of a schedule determined for two probes for this exemplary system in accordance with the above described schedule planning method according to the invention can be found in FIG. 11. The schedule determines specifically which task is performed for which probe by which of the equipment, i.e. units, of the high throughput equipment.

A further preferred application of the invention refers to determining and controlling a schedule for a formulation and/or synthesis robot. Such a robot can be configured to provide a plurality of functions needed for the synthesis of molecular formulations, for example, the robot can be configured to add or remove substances from a probe container, shake or vibrate a probe, apply different measurement and analysis tools to a probe, etc. A task to be performed on a probe can then refer to synthesising a substance based on a recipe or synthesis specification determining operations to be performed on a base material probe. Generally, such a robot can provide different restrictions to a synthesis schedule that depend on the specific construction of the robot. For example, a robot can be strongly restricted with respect to how often each operation type can be performed on a probe or can be restricted to a certain sequence of possible operations. Moreover, the robot can also be restricted on how many probes an operation can be performed at the same time. Furthermore, the construction of the robot can determine whether the robot can perform different operations in parallel or only sequentially. For this preferred application it is thus preferred that the technical information of the robot indicating the respective constraints of an individual robot are taken into account in the formulation of the scheduling problem, for example, as described above.

In a further preferred application the invention refers to determining and controlling a schedule for long term stability units. In this case the tasks refer to long term analyzing of probes that are stored for a long time, for instance, days, weeks, or even years, in between different testing procedures. For such scenarios automated systems allow a management for storing and retrieving the probes, for instance, provided in probe trays, based on a predetermined schedule. In this case the above described invention allows to optimize the schedule for each probe in the long term testing procedure, in particular, such that the laboratory equipment utilized for the testing procedure performed on retrieved probes is effective utilized, in particular, with as few down times as possible. An example of a long term stability facility and possible associated equipment, i.e. units, can be found in FIG. 12 together with exemplary capacities for each station of the long term stability unit. The station exemplarily shown in FIG. 12 refers to a storage unit, a transport system and a measurement unit, wherein only one probe tray at a time can be transferred from the storage to the measurement unit.

In a further preferred application the invention refers to determining and controlling a schedule for laboratory diagnostic processes, like PCR processes. In this case, the procedure can be performed with respective automated or semi-automated laboratory equipment dedicated to the respective tasks.

Generally, it is one of the main goals of the optimizing of the schedule to allow for an optimal execution of the operations of one or more tasks associated with one or more probes while avoiding deadlocks. This, allows to use the full capacity of automated or semi-automated laboratory systems. However, for providing also accurate results for each task, it is preferred that also the timing of respective operations associated with the tasks is taken into account, for instance, respective waiting times between different operations on a probe. The apparatus, for example, as described above, in the following also called an object scheduler, is preferably configured to control and organize the tasks in a batch production or continuous production manner. In case of a batch production manner a substantial fixed probe list is provided that forms a current batch and after the optimization the respective determined schedule is completed before starting a new batch process with a new probe list. In case of a continuous production manner, a probe list can be variable, in particular, new probes can be added to the probe list and already processed probes are removed from the probe list. In this case the scheduler is preferably adapted, as already described exemplarily above, to update the probe list and to determine a schedule for the updated probe list such that the controlling of the automated processes is continuously adapted to the added or removed probes.

As input to the scheduler, for example, via the probe list providing unit described above, a probe list is provided comprising probe identifications and tasks associated with a respective probe. The task is indicative for the operations that are to be performed by the respective laboratory equipment on the probe and also for the intended timing of the operations. The scheduler can then determine the schedule for the probe list as described, for example, with respect to any of FIGS. 7 to 9. If the probes are processed in a continuous process, during the performing of the already determined schedule one or more new probes can be added arbitrarily, and the scheduler is preferably adapted to perform a rescheduling based on an updated probe list.

Generally, the scheduler, in particular, the scheduling problem formulation unit as described above, is adapted to formulate a scheduling problem based on the provided probe list, in particular, based on the provided probe identifications and associated tasks. In a preferred example, the scheduling problem can be formulated as an integer or mixed integer problem with an objective function referring to minimizing the time needed to perform all tasks of the probe list.

Further, it is preferred that the scheduler, in particular the scheduling problem formulation unit, is adapted to take constraints into account when formulating the scheduling problem. The constraints can refer to technical constraints of the laboratory equipment, like a processing capacity, or a task constraint, like necessary waiting times between different operations of a task. These constraints can be determined, for example, from the probe list, in particular, from the tasks provided by the probe list and/or based on additional information, like technical information on the laboratory equipment. Generally, the scheduling problem can be formulated with categorical variables or with continuous variables. However, it is preferred that the scheduling problem is formulated using categorical variables. If continuous variables are utilized it is preferred the scheduling problem is transformed into categorical variables before being provided to the quantum computer. For instance, the problem preparation unit as described above can be adapted to reformulate the scheduling problem accordingly, or to determine parts of the scheduling problem, i.e. subproblems, that can be reformulated using categorical variables. Utilizing categorical variables allows to utilize as quantum computer a Futjisu Annealer or D-Wave Annealer. Preferably, the scheduler, for example, utilizing the scheduling problem formulation unit or the problem preparation unit, is adapted to formulate the scheduling problem using binary variables.

Preferably, also the constraints as mentioned above are taken into account in the formulation of the scheduling problem. In a preferred example, the constraints are taken into account as part of the scheduling problem by providing the scheduling problem with additional conditions that have to be fulfilled during the optimization. In another preferred embodiment the constraints are taken into account as part of the scheduling problem by adding the constraints as penalty terms to the objective function of the scheduling problem. In this case the constraints are directly taken into account during the optimization of the objective function. Moreover, this embodiment has the advantage that the constraints can be weighted individually. For example, different constraints can be weighted with different weights such that, for instance, also an importance that a constraint is met can be reflected during the optimization. In the following an example on how constraints can be taken into account is provided. In this example, at least one of the laboratory equipment resources allows to process two probes in parallel in the same time slot. Thus, this constraint can be provided as additional condition in the scheduling problem, wherein the additional condition can mathematically be formulated as a sum over all probes processed by the respective resource in a time slot, wherein this sum has to be lower or equal than two for all solutions of the objective function. In the alternative preferred example this constraint can be formulated as penalty term in the objective function. For example, mathematically the constraint can be formulated as sum over all probes processed by the respective resource in a time slot minus two and then added to the objective function of the scheduling problem. Moreover, this term can then also be taken as quadratic term in order to increase the penalty for solutions diverging from the intended constraint. Moreover, a weight can be provided for weighting this penalty term during the optimization of the objective function. Further constraints that are preferably taken into account when formulating the scheduling problem can refer to sequence constraints, like which operation has to be performed first, time constraints, like waiting times between operations, task dependencies, like tasks that can only be performed when other tasks are already finished, capacity constraints, like the amount of probes that can be processed in parallel in a time slot, optional tasks, like clean-up tasks or storage tasks, etc.

Other variations to the disclosed embodiments can be understood and effected by those skilled in the art in practicing the claimed invention, from a study of the drawings, the disclosure, and the appended claims.

For the processes and methods disclosed herein, the operations performed in the processes and methods may be implemented in differing order. Furthermore, the outlined operations are only provided as examples, and some of the operations may be optional, combined into fewer steps and operations, supplemented with further operations, or expanded into additional operations without detracting from the essence of the disclosed embodiments.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the providing of the probe list, the formulation of the scheduling problem, the interfacing with the quantum computer, the determining of the schedule, etc. performed by one or several units or devices can be performed by any other number of units or devices. These procedures can be implemented as program code means of a computer program and/or as dedicated hardware.

A computer program product may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would understand that the structure of an executable component may include software objects, routines, methods, and so forth, that may be executed on the computing system. This may include both an executable component in the heap of a computing system, or on computer-readable storage media. The structure of the executable component may exist on a computer-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other instances, structures may be hard coded or hard wired logic gates, that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communications connection, for example, either hardwired, wireless, or a combination of hardwired or wireless, to a computing system, the computing system properly views the connection as a transmission medium. Transmission media can include a network and/or data links which can be used to carry desired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

The invention refers to an apparatus for optimizing a laboratory scheduling control. A providing unit provides a probe list including probe data. The probe data comprises probe identifications and tasks. A task is indicative of operations to be performed by a laboratory equipment on the probe. A formulation unit formulates a scheduling problem based on the probe list such that an optimization of the scheduling problem results in an optimization of a scheduling of the tasks. An interface unit interfaces with a quantum computer for utilizing quantum computing for optimizing the scheduling problem and receiving a result of the quantum computation indicative of the optimized scheduling problem. A determination unit determines a schedule for the probe list based on the received result. A control signal determination unit determines a control signal for controlling the laboratory equipment based on the determined schedule.

1. An apparatus for optimizing a laboratory scheduling control, wherein the apparatus comprises:
a probe list providing unit for providing a probe list including probe data for a plurality of probes, wherein the probe data for a probe comprises a probe identification and a task associated with the probe identification, wherein a task is indicative of one or more operations to be performed with a predetermined timing by a laboratory equipment on the probe,
a scheduling problem formulation unit for formulating a scheduling problem based on the probe list, wherein the scheduling problem is formulated such that an optimization of the scheduling problem results in an optimization of a scheduling of the tasks associated with the plurality of probes,
a quantum computer interface unit for interfacing with a quantum computer for utilizing quantum computing for optimizing the scheduling problem and receiving a result of the quantum computation from the quantum computer indicative of the optimized scheduling problem,
a schedule determination unit for determining a schedule for the probe list based on the received result of the quantum computation, and
a control signal determination unit for determining a control signal for controlling the laboratory equipment based on the determined schedule.

2. The apparatus according to claim 1, wherein the quantum computer interface unit further comprises a scheduling problem preparation unit for preparing at least a part of the scheduling problem such that the optimization of the scheduling problem is performable utilizing a quantum computation.

3. The apparatus according to claim 2, wherein the preparation of the scheduling problem comprises determining control operations for controlling the quantum computer to perform the quantum computation of at least a part of the scheduling problem and wherein the scheduling problem preparation unit is further adapted to send the control operations to the quantum computer for performing the quantum computation of the at least a part of the scheduling problem.

4. The apparatus according to claim 2, wherein the scheduling problem preparation unit is adapted to prepare the scheduling problem to be solvable on a quantum computer that refers to a quantum annealer or that refers to a gate-based quantum computer.

5. The apparatus according to claim 1, wherein the scheduling problem is formulated such that an optimization of the scheduling problem refers to an optimization of the time needed for performing operations indicated by the tasks.

6. The apparatus according to claim 1, wherein the apparatus further comprises a constraints determination unit for determining constraints for the scheduling based on the tasks and/or based on laboratory equipment information indicative of technical data of the laboratory equipment, wherein the scheduling problem is formulated further based on the determined constraints.

7. The apparatus according to claim 6, wherein the laboratory equipment information is further indicative on whether one or more operations on one or more probes are performable at the same time or sequentially, wherein the constraints are further determined based on this information.

8. The apparatus according to claim 1, wherein the probe list providing unit is further adapted to provide an updated probe list based on probe data received after the determination of the schedule for the original probe list, wherein the scheduling problem formulation unit is then adapted to reformulate the scheduling problem based on the updated probe list to determine an updated scheduling.

9. A quantum computer system comprising:
an interface unit for receiving, from the apparatus according to claim 1, a scheduling problem formulated such that an optimization of the scheduling problem results in an optimization of a scheduling of tasks associated with a plurality of laboratory probes, wherein the task are to be performed by laboratory equipment on the plurality of laboratory probes, and
a quantum computer adapted to perform a quantum computation of the received scheduling problem,
wherein the interface unit is further adapted to provide a result of a quantum computation of the scheduling problem to the apparatus for further processing.

10. A laboratory system comprising:
laboratory equipment adapted to perform one or more operations indicated by tasks in an automatic manner on one or more laboratory probes based on control signals, and
an apparatus according to claim 1, wherein the apparatus provides the determined control signals to the laboratory equipment to perform the determined schedule.

11. A scheduling determination system comprising:
an apparatus according to claim 1, and
a quantum computer system, wherein the apparatus and the quantum computer system are connected via the interface units, and wherein the quantum computer system comprises
an interface unit for receiving, from the apparatus according to claim 1, a scheduling problem formulated such that an optimization of the scheduling problem results in an optimization of a scheduling of tasks associated with a plurality of laboratory probes, wherein the task are to be performed by laboratory equipment on the plurality of laboratory probes, and
a quantum computer adapted to perform a quantum computation of the received scheduling problem,
wherein the interface unit is further adapted to provide a result of a quantum computation of the scheduling problem to the apparatus for further processing.

12. A method for optimizing a laboratory scheduling control, wherein the method comprises:
providing a probe list including probe data for a plurality of probes, wherein the probe data for a probe comprises a probe identification and a task associated with the probe identification indicative of one or more operations to be performed with a predetermined timing by a laboratory equipment on the probe,
formulating a scheduling problem based on the probe list, wherein the scheduling problem is formulated such that an optimization of the scheduling problem results in an optimization of a scheduling of the tasks associated with the plurality of probes,
sending, via an interface, the scheduling problem to the quantum computer to be optimized utilizing quantum computing and receiving, via the interface, a result of the optimization of the scheduling problem from the quantum computer,
determining a schedule for the probe list based on the received result of the quantum computation, and
determining a control signal for controlling the laboratory equipment based on the determined schedule.

13. A computer program product for optimizing a laboratory scheduling control, wherein the computer program product comprises program code means for causing apparatus to execute the method according to claim 12, wherein the apparatus comprises:
a probe list providing unit for providing a probe list including probe data for a plurality of probes, wherein the probe data for a probe comprises a probe identification and a task associated with the probe identification, wherein a task is indicative of one or more operations to be performed with a predetermined timing by a laboratory equipment on the probe,
a scheduling problem formulation unit for formulating a scheduling problem based on the probe list, wherein the scheduling problem is formulated such that an optimization of the scheduling problem results in an optimization of a scheduling of the tasks associated with the plurality of probes,
a quantum computer interface unit for interfacing with a quantum computer for utilizing quantum computing for optimizing the scheduling problem and receiving a result of the quantum computation from the quantum computer indicative of the optimized scheduling problem,
a schedule determination unit for determining a schedule for the probe list based on the received result of the quantum computation, and
a control signal determination unit for determining a control signal for controlling the laboratory equipment based on the determined schedule.

14. A scheduling problem preparation apparatus comprising:
an interface for receiving a scheduling problem, wherein the scheduling problem is formulated such that an optimization of the scheduling problem results in an optimization of a scheduling of tasks associated with a plurality of probes,
a processor configured for preparing at least a part of the scheduling problem such that the optimization of the scheduling problem is performable utilizing a quantum computation, and
an interface for providing the prepared at least a part of the scheduling problem to a quantum computer for calculating the prepared at least a part of the scheduling problem.
